(19) United States
(12) Patent Application Publication
(10) Pub. No.: US 2007/0088073 A1
(43) Pub. Date: Apr. 19, 2007

(54) METHOD FOR TREATING PAIN

(75) Inventors: Vincenzo Di Marzo, Pozzuoli (IT); Luciano De Petrocellis, Pozzuoli (IT); Sabatino Malone, Brusciano-Naples (IT); Vito De Novellis, S. Maria Capua Vetere (IT)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 11/549,276

(22) Filed: Oct. 13, 2006

Related U.S. Application Data
(60) Provisional application No. 60/728,851, filed on Oct. 19, 2005.

(51) Int. Cl. A61K 31/405; C07D 209/18

(52) U.S. Cl. 514/419; 548/495

(57) ABSTRACT

The present invention provides pharmaceutical compositions useful in a method for treating neuropathic pain, said method comprising administration of a pain-ameliorating effective amount of the compound according to formula I 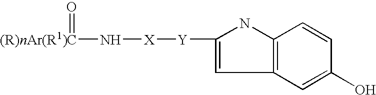 wherein R is an alk(en)yl group, R1 is an alkylen(yl) group, n is 0 or 1 and Ar is a carbocyclic aryl group;

wherein X is —CH₂—, —CH—, or 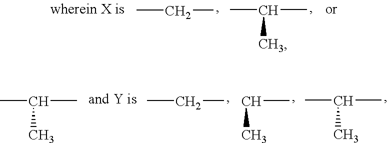 O, S, or NR wherein R is H or

(CH₂)ₘH and m is an integer of from 1 to 5 or the bond between X and Y may be a double or triple bond, e.g. as in X═Y or X≡Y.

### BACKGROUND OF THE INVENTION

1. Field of the Invention

The present invention relates to the treatment or prevention of pain or nociception.

2. Related Art

Pain is a sensory experience distinct from sensations of touch, pressure, heat and cold. It is often described by sufferers by such terms as bright, dull, aching, pricking, cutting or burning and is generally considered to include both the original sensation and the reaction to that sensation. This range of sensations, as well as the variation in perception of pain by different individuals, renders a precise definition of pain difficult, however, many individuals suffer with severe and continuous pain.

Pain that is caused by damage to neural structures is often manifest as a neural supersensitivity or hyperalgesia and is termed “neuropathic” pain. Pain can also be “caused” by the stimulation of nociceptive receptors and transmitted over intact neural pathways, such pain is termed “nociceptive” pain.

The level of stimulation at which pain becomes noted is referred to as the “pain threshold.” Analgesics are pharmaceutical agents which relieve pain by raising the pain threshold without a loss of consciousness. After administration of an analgesic drug a stimulus of greater intensity or longer duration is required before pain is experienced. In an individual suffering from hyperalgesia an analgesic drug may have an anti-hyperalgesic effect. In contrast to analgesics, agents such as local anaesthetics block transmission in peripheral nerve fibers thereby blocking awareness of pain. General anaesthetics, on the other hand, reduce the awareness of pain by producing a loss of consciousness.

### BRIEF SUMMARY OF THE INVENTION

It has now been discovered that certain compounds which exhibit the properties of blocking transient receptor potential vanilloid type 1 (TRPV1) channels and activating cannabinoid CB₁ receptors have a utility for the amelioration of pain and particularly for the amelioration of neuropathic pain. The compounds of the invention are analogues and/or homologues of the compound N-arachidonyl-serotonin (A-5-HT).

Therefore, in one aspect, the method of the present invention utilizes analogues and/or a homogue of the a N-alk(en)yl carbocyclic aryl alk(yl)enyl carbo-serotonin adduct to treat pain. The compounds utilized in the method of the present invention are amides of hydrocarbyl acids and an analogue and/or a homologue of serotonin wherein said hydrocarbyl moiety includes enchained aryl radicals. That is, the compounds are N-(alkyl)ₙ carbocyclic aryl alkanoyl-serotonin compounds wherein n is 0 or 1 and said (alkyl)ₙ carbocyclic aryl alkanoyl radical includes from 7 to 30 carbon atoms, e.g. from 8 to 22 carbon atoms. Said alkyl group and said alkanoyl group may include 1 or more, e.g. 1-3 unsaturated bonds. That is, said alkyl group may be an alkenyl group, including a conjugated alkenyl group. Thus, the designation as alk(en)yl is utilized. Said alkanoyl group may be an alkenoyl group. Thus, the designation alk(yl)enyl carbo is utilized. Said compound may be represented by the formula I

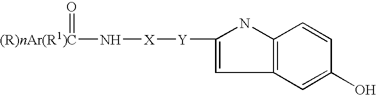

wherein R is an alk(en)yl group, R¹ is an alkylen(yl) group, n is 0 or 1, Ar is a carbocyclic aryl group;

wherein X is —CH₂—, —CH—, or

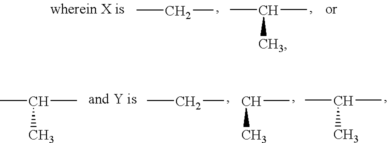

O, S, or NR wherein R is H or

(CH₂)ₘH and m is an integer

of from 1 to 5, e.g. 1 or 2.

Alternatively, the bond between X and Y may be a double or triple bond, e.g. as in X═Y or X≡Y.

Preferably R is an alkyl or alkenyl group comprising from 1 to 7 carbon atoms and more preferably R is an methyl alkylene or methyl alkenyl group such as CH₃CH₂, CH₃CH₂CH₂, CH₃CH₂CH₂CH₂, CH₃CHCH, or CH₃CHCHCH₂CHCHCH₂.

Preferably R¹ is an alkylene group consisting from 3 to 6 carbon atoms, such as CH₂CH₂CH₂, CH₂CH₂CH₂CH₂, CH₂CH₂CH₂CH₂CH₂CH₂, etc.

Preferably Ar is selected from the group consisting of phenyl, naphthyl and biphenyl and lower alkyl substituted derivatives thereof, i.e. C₁ to C₄ alkyl substituted derivatives thereof.

For example, the compounds of formula I may be selected from the group consisting of

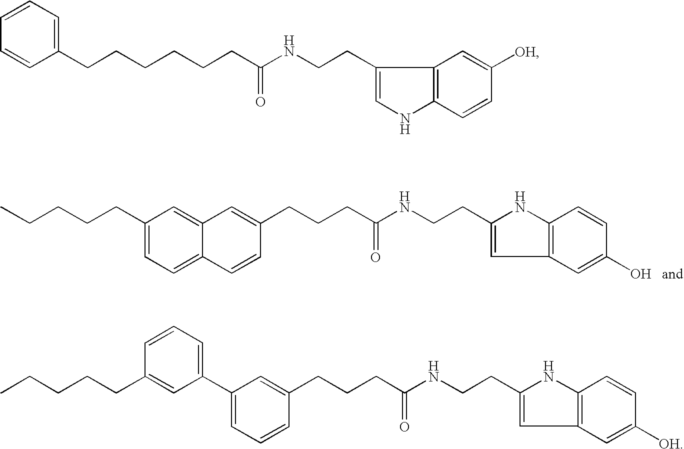

The compounds of this invention may be prepared by reacting serotonin or an analogue or homologue thereof with a carboxylic acid to form the corresponding amide of said carboxylic acid and serotonin (or analogue or homologue thereof). This reaction may be carried out at conditions known in the art for preparing amides of fatty acids e.g., which fatty acids have similar reaction properties as the above carboxylic acids.

In another aspect, the invention provides a method for the treatment of pain using a compound in accord with formula I, the method comprising administering a pain-ameliorating effective amount of the compound.

In another embodiment, the method comprises administration of a pain-ameliorating effective amount of a compound according to formula I in the form of a pharmaceutical composition comprising a compound according to formula I as an active ingredient together with one or more pharmaceutically-acceptable additives.

In a further embodiment, the method comprises binding a compound according to formula I to the TRPV1 channel of a warm-blooded animal, such as a human being, so as to beneficially inhibit the activity of said channel to activation by capsaicin, for example.

In a further embodiment, the method comprises binding a compound according to formula I to the fatty acid amide hydrolase of a warm-blooded animal, such as a human being, so as to enhance endocannabinoid levels and activate cannabinoid receptors m said animals to thereby ameliorate pain.

Yet other aspects of the invention are pharmaceutical compositions which contain the compound in accord with formula I and the use of the compound in accord with formula I for the preparation of medicaments and pharmaceutical compositions

### DETAILED DESCRIPTION OF THE INVENTION

Genetic or pharmacological targeting of fatty acid amide hydrolase (FAAH), one of the enzymes catalysing endocannabinoid degradation, was shown to result in analgesic and anti-hyperalgesic actions that are due to the “indirect” activation (via enhancement of endocannabinoid levels) of cannabinoid CB₁ receptors. Additionally, genetic or pharmacological targeting of transient receptor potential vanilloid type 1 (TRPV1) channels was found to abolish thermal and inflammatory analgesia. We describe a class of “hybrid” FAAH inhibitors/TRPV1 antagonists with high efficacy against inflammatory hyperalgesia. These “hybrid” FAAH inhibitors are homologues and/or analogues of AA-5-HT and have the general formula I:

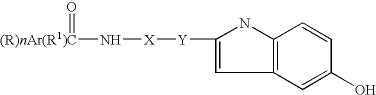

wherein R is an alk(en)yl group, R¹ is an alkylen(yl) group, n is 0 or 1 and Ar is a carbocyclic aryl group;

wherein X is —CH₂—, —CH—, or

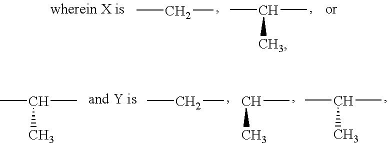

O, S, or NR wherein R is H or

(CH₂)ₘH and in is an integer

of from 1 to 5, e.g. 1 or 2.

Alternatively, the bond between X and Y may be a double or triple bond, e.g. as in X═Y or X≡Y.

Preferably R is an alkyl or alkenyl group comprising from 1 to 7 carbon atoms and more preferably R is an methyl alkylene or methyl alkenyl group such as CH₃CH₂, CH₃CH₂CH₂, CH₃CH₂CH₂CH₂, CH₃CHCH, or CH₃CHCHCH₂CHCHCH₂.

Preferably R¹ is an alkylene group consisting from 3 to 6 carbon atoms, such as CH₂CH₂CH₂, CH₂CH₂CH₂CH₂, CH₂CH₂CH₂CH₂CH₂CH₂ etc.

Preferably Ar is selected from the group consisting of phenyl, naphthyl and biphenyl and lower alkyl substituted derivatives thereof, i.e. C₁ to C₄ alkyl substituted derivatives thereof.

For example, the compounds of formula I may be selected from the group consisting of

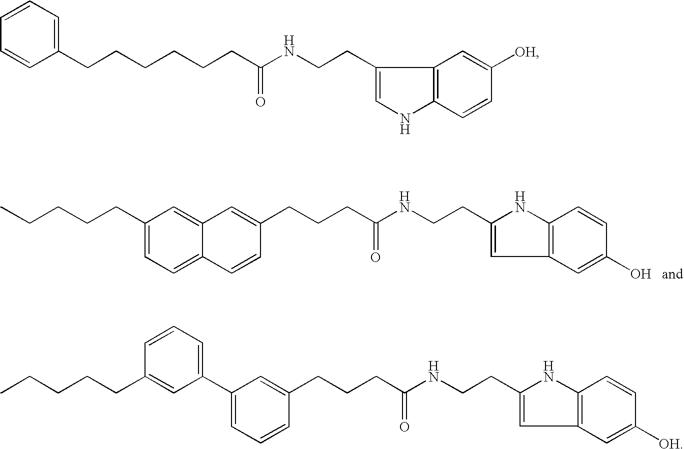

These compounds inhibit FAAH and, also interact, by blocking their activation by capsaicin, with TRPV1 channels, whose gating plays a permissive role in the development of hyperlagesia.

When injected directly into the periaqueductal grey (PAG) of rats, the compounds of this invention potently inhibit both phases of the nociceptive response to formalin injected into the rat paw and concomitantly elevate anandamide levels in this area of the brainstem. The effect is counteracted by the CB₁ receptor antagonist, AM251 (nmol/rat) and is occluded by the TRPV1 antagonist, capsazepine (6 nmol/rat). Thus, while not wishing to be bound by theory, it is believed that the compounds of formula I ameliorate pain by the dual mechanism of action of both “indirect” activation of CB₁ and antagonism of TRPV1. The compound acts at the supraspinal level by blocking the inhibitory effect of formalin on the OFF cells of the rostral ventromedial medulla, which receive synapses with cells from the PAG. Also this effect is reversed by AM251 and occluded by capsazepine. When injected into the paw, these compounds, selectively block the 2^{nd}, inflammatory phase of the nocifensive response to formalin, again in a way counteracted by AM251 and occluded by capsazepine, thus suggesting also a peripheral mode of action. The compounds are novel agents against anti-inflammatory pain, acting by enhancing endocannabinoid levels (via FAAH inhibition) and at the same time by antagonizing TRPV1.

The advantage of having in one molecule a FAAH inhibitor and a TRPV1 antagonist comes from the several experimental observations suggesting that FAAH inhibitors (i.e. “indirect” agonists of cannabinoid and fatty acid amide receptors) as well as direct cannabinoid receptor agonists (both CB1 and CB2) are very promising against inflammatory and neuropathic pain, and so are compounds that block TRPV1 receptors. However, different populations of neurons/cells and different mechanisms are involved in CB1/CB2- and TRPV1-mediated anti-inflammatory and anti-hyperalgesic/anti-allodynic effects. Therefore, if for example following nerve injury, only one of these different populations is destroyed, a compound only acting on that population will be ineffective, whereas a compound with “hybrid” activity will always be more effective. On the other hand if different nociceptive mechanisms cause pain, a drug targeting more of these mechanisms will be more efficacious than a drug specific for only one of them.

To use the compound of the invention or a pharmaceutically-acceptable salt thereof for the therapeutic treatment, which may include prophylactic treatment, of pain in mammals, which may be humans, the compound can be formulated in accordance with standard pharmaceutical practice as a pharmaceutical composition.

Suitable pharmaceutical compositions that contain the compounds of the invention may be administered in conventional ways, for example by oral, topical, parenteral, buccal, nasal, vaginal or rectal administration or by inhalation. For these purposes a compound of the invention may be formulated by means known in the art into the form of, for example, tablets, capsules, aqueous or oily solutions, suspensions, emulsions, creams, ointments, gels, nasal sprays, suppositories, finely divided powders or aerosols for inhalation, and for parenteral use (including intravenous, intramuscular or infusion) sterile aqueous or oily solutions or suspensions or sterile emulsions. A preferred route of administration is orally by tablet or capsule

In addition to a compound of the present invention a pharmaceutical composition of this invention may also contain one or more other pharmacologically-active agents, or such pharmaceutical composition may be simultaneously or sequentially co-administered with one or more other pharmacologically-active agents.

Pharmaceutical compositions of this invention will normally be administered so that a pain-ameliorating effective daily dose is received by the subject. The daily dose may be given in divided doses as necessary, the precise amount of the compound received and the route of administration depending on the weight, age and sex of the patient being treated and on the particular disease condition being treated according to principles known in the art. A preferred dosage regime is once daily.

A further embodiment of the invention provides a pharmaceutical composition which contains a compound of the invention as defined herein or a pharmaceutically-acceptable salt thereof, in association with a pharmaceutically-acceptable additive such as an excipient or canter.

A yet firmer embodiment of the invention provide the use of the compound of the invention, or a pharmaceutically-acceptable salt thereof, in the manufacture of a medicament useful for blocking the TRPV1 channel in a warm-blooded animal such as a human being.

Still another embodiment of the invention provides a method of binding the compound of the invention to the TRPV1 channel of a warm-blooded animal, such as a human being, in need of treatment for pain, which method comprises administering to said animal an effective amount of a compound of formula I or a pharmaceutically-acceptable salt thereof.

A yet farther embodiment of the invention comprises the use of the compound of the invention, or a pharmaceutically-acceptable salt thereof in the manufacture of a medicament useful for activating the cannabinoid CB₁ receptor in a warm-blooded animal such as a human being.

Still another embodiment of the invention provides a method of binding the compound of the invention to the fatty acid amide hydrolase of a warm-blooded animal, such as a human being, in need of treatment for pain, which method comprises administering to said animal an effective amount of a compound of formula I or a pharmaceutically-acceptable salt thereof.

The foregoing description details specific methods and compositions tat can be employed to practice the present invention, and represents the best mode contemplated. Thus, however detailed the foregoing may appear in text, it should not be construed as limiting the overall scope hereof; rather, the ambit of the present invention is to be governed only by the lawful construction of the appended claims.

What is claimed is:

1. A pharmaceutical composition comprising, as the active compound, a compound represented by formula I 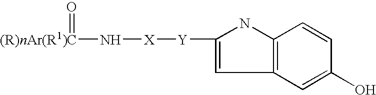
wherein R is an alk(en)yl group, R¹ is an alkylen(yl) group, n is 0 or 1 and Ar is a carbocyclic aryl group;
wherein X is —CH₂—, —CH—, or 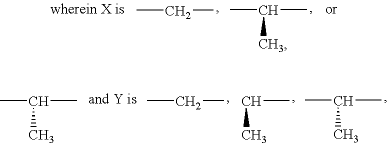
O, S, or NR wherein R is H or
(CH₂)ₘH and m is an integer
of from 1 to 5, or
the bond between X and Y is a double or triple bond.

2. The composition of claim 1 wherein m is 1 or 2.

3. The composition of claim 2 wherein R is an alkyl or alkenyl group comprising from 1 to 7 carbon atoms and R¹ is an alkylene comprising from 3 to 6 carbon atoms and Ar is selected from the group consisting of phenyl, naphthyl and biphenyl and lower alkyl substituted derivatives thereof.

4. The composition of claim 3 wherein R is selected from the group consisting of CH₃CH₂, CH₃CH₂CH₂, CH₃CH₂CH₂CH₂, CH₃CHCH and CH₃CHCHCH₂CHCHCH₂ and R¹ is selected from the group consisting of CH₂CH₂CH₂, CH₂CH₂CH₂CH₂ and CH₂CH₂CH₂CH₂CH₂CH₂.

5. A method for treating neuropathic pain, said method comprising administration of a pain-ameliorating effective amount of a compound represented by formula I 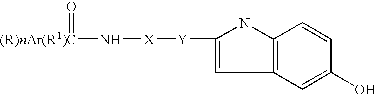
wherein R is an alk(en)yl group, R¹ is an alkylen(yl) group, n is 0 or 1 and Ar is a carbocyclic aryl group;
wherein X is —CH₂—, —CH—, or 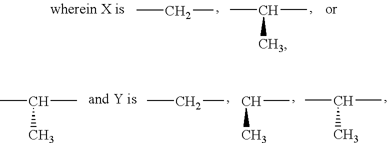
O, S, or NR wherein R is H or
(CH₂)ₘH and m is an integer
of from 1 to 5 or
the bond between X and Y may be a double or triple bond.

6. The method of claim 5 wherein R is an alkyl or alkenyl group comprising from 1 to 7 carbon atoms and R¹ is an alkylene comprising from 3 to 6 carbon atoms and Ar is selected from he group consisting of phenyl, naphthyl and biphenyl and lower alkyl substituted derivatives thereof.

7. The method of claim 6 wherein R is selected from the group consisting of CH₃CH₂, CH₃CH₂CH₂, CH₃CH₂CH₂CH₂, CH₃CHCH and CH₃CHCHCH₂CHCHCH₂ and R¹ is selected from the group consisting of CH2CH₂CH₂, CH₂CH₂CH₂CH₂ and CH₂CH₂CH₂CH₂CH₂CH₂.

8. A method comprising binding a compound to the TRPV1 channel of a warm-blooded animal, such as a human being, so as to beneficially inhibit the activity of said channel to thereby ameliorate pain wherein said compound is represented by formula I 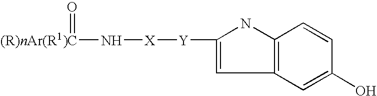
wherein R is an alk(en)yl group, R¹ is an alkylen(yl) group, n is 0 or 1 and Ar is a carbocyclic aryl group;
wherein X is —CH₂—, —CH—, or 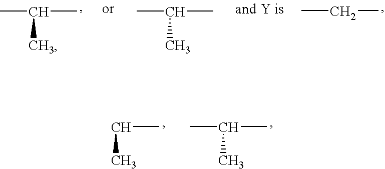
O, S, or NR wherein R is H or
(CH₂)ₘH and m is an integer
of from 1 to 5 or
the bond between X and Y may be a double or triple bond.

9. The method of claim 8 wherein R is an alkyl or alkenyl group comprising from 1 to 7 carbon atoms and R¹ is an alkylene comprising from 3 to 6 carbon atoms and Ar is selected from the group consisting of phenyl, naphthyl and biphenyl and lower alkyl substituted derivatives thereof.

10. The method of claim 9 wherein R is selected from the group consisting of CH₃CH₂, CH₃CH₂CH₂, CH₃CH₂CH₂CH₂, CH₃CHCH and CH₃CHCHCH₂CHCHCH₂ and R¹ is selected from the group consisting of CH₂CH₂CH₂, CH₂CH₂CH₂CH₂ and CH₂CH₂CH₂CH₂CH₂CH₂.

11. A method comprising binding a compound to the fatty acid amide hydrolase of a warm-blooded animal, such as a human being, so as to activate said receptor to enhance endocannabinoid levels in said animal to thereby ameliorate pain, wherein said compound is represented by formula I 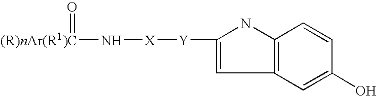
wherein R is an alk(en)yl group, R¹ is an alkylen(yl) group, n is 0 or 1 and Ar is a carbocyclic aryl group;
wherein X is —CH₂—, —CH—, or 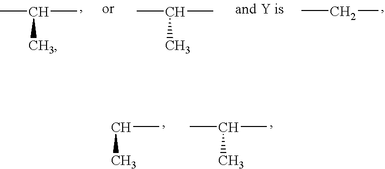
O, S, or NR wherein R is H or
(CH₂)ₘH and m is an integer
of from 1 to 5 or
the bond between X and Y is a double or triple bond,

12. The method of claim 11 wherein R is an alkyl or alkenyl group comprising from 1 to 7 carbon atoms and R¹ is an alkylene comprising from 3 to 6 carbon atoms and Ar is selected from the group consisting of phenyl, naphthyl and biphenyl and lower alkyl substituted derivatives thereof.

13. The method of claim 12 wherein R is selected from the group consisting of CH₃CH₂, CH₃CH₂CH₂, CH₃CH₂CH₂CH₂, CH₃CHCH and CH₃CHCHCH₂CHCHCH₂ R¹ is selected from the group consisting of CH₂CH₂CH₂, CH₂CH₂CH₂CH₂ and CH₂CH₂CH₂CH₂CH₂CH₂.

14. A compound according to formula I 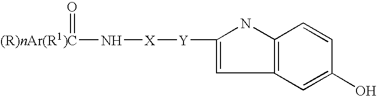
wherein R is an alk(en)yl group, R¹ is an alkylen(yl) group, n is 0 or 1 and Ar is a carbocyclic aryl group;
wherein X is —CH₂—, —C—, or 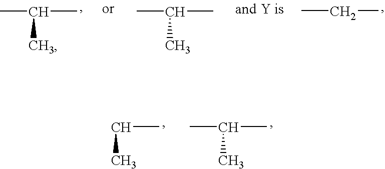
O, S, or NR wherein R is H or
(CH₂)ₘH and m is an integer
of from 1 to 5 or
the bond between X and Y may be a double or triple bond, e.g. as in X═Y or X≡Y.

15. The compound of claim 14 wherein R is an alkyl or alkenyl group comprising from 1 to 7 carbon atoms, R is an methyl alkylene or methyl alkenyl group selected from the group consisting of R¹ is an alkylene group consisting from 3 to 6 carbon atoms and Ar is selected from the group consisting of phenyl, naphthyl and biphenyl and C₁ to C₄ alkyl substituted derivatives thereof

16. The compound of claim 15 wherein R is selected from the group consisting of CH₂CH₂CH₂, CH₂CH₂CH₂CH₂, CH₂CH₂CH₂CH₂CH₂CH₂, and R¹ is selected from the group consisting of R is an methyl alkylene and methyl alkenyl group such as CH₃CH₂, CH₃CH₂CH₂, CH₃CH₂CH₂CH₂, CH₃CHCH and CH₃CHCHCH₂CHCHCH₂.

17. A method of treating neuropathic pain, said method comprising administration of a pain-ameliorating amount of the composition of claim 1 to a warm-blooded animal to thereby bind the compound to the TRPV1 channel so as to beneficially inhibit the activity of said channel and activate the cannabinoid receptor to enhance endocannabinoid levels in said animal to thereby ameliorate pain.
